(19) United States
(12) Patent Application Publication
(10) Pub. No.: US 2001/0004779 A1
(43) Pub. Date: Jun. 28, 2001

(54) Novel p-aminophenol derivatives and the use thereof

(76) Inventors: David Rose, Hilden (DE); Bernd Meinigke, Leverkusen (DE); Horst Hoeffkes, Duesseldorf (DE)

Correspondence Address: Kimberly R. Hild, Henkel Corporation, Patent Law Department, 2500 Renaissance Boulevard,Suite 200, Gulph Mills, PA, 19406, US

(21) Appl. No.: 09/783,874

(22) Filed: Feb. 15, 2001

Related U.S. Application Data
(63) Continuation of application No. 09/581,034, filed on Oct. 2, 2000, now abandoned.
Application No. 09/581,034 is a 371 of international application No. PCT/EP98/07961, filed on Dec. 8, 1998.

(30) Foreign Application Priority Data
Dec. 17, 1997 (DE) 197 56 137.3

(51) Int. Cl. A61K 7/13

(52) U.S. Cl. 8/416; 8/405

(57) ABSTRACT

The present invention relates to new p-aminophenol derivatives and to methods of using the same for coloring keratin fibers. The present invention also relates to colorant compositions containing these compounds.

### CROSS REFERENCE TO RELATED APPLICATIONS

This application is a continuation of application Ser. No. 09/581,034 filed on Oct. 2, 2000, which is a 371 application of PCT EP98/07961 filed on Dec. 8, 1998.

### FIELD OF THE INVENTION

This invention relates to new p-aminophenol derivatives, to their use for coloring keratin fibers and to colorants containing these compounds.

### BACKGROUND OF THE INVENTION

By virtue of their intensive colors and good fastness properties, so-called oxidation colorants play a prominent role in the coloring of keratin fibers, particularly human hair. Oxidation colorants contain oxidation dye precursors, so-called primary intermediates and secondary intermediates. The primary intermediates form the actual dyes with one another or by coupling with one or more secondary intermediates in the presence of oxidizing agents or atmospheric oxygen.

Good oxidation dye precursors are expected to satisfy above all the following requirements: they must form the required color tones with sufficient intensity and fastness during the oxidative coupling reaction. In addition, they must be readily absorbed onto the fibers with no significant differences —particularly in the case of human hair —between damaged and freshly regrown hair (levelling behavior). They must be resistant to light, heat and the effect of chemical reducing agents, for example permanent wave lotions. Finally, if they are used to color hair, they should not overly stain the scalp and, above all, should be toxicologically and dermatologically safe.

The primary intermediates normally used are primary aromatic amines containing another free or substituted hydroxy or amino group in the para position or the ortho position, diaminopyridine derivatives, heterocyclic hydrazones, 4-aminopyrazolone derivatives and 2,4,5,6-tetraaminopyrimidine and derivatives thereof.

Special representatives are, for example, p-phenylenediamine, p-toluylenediamine, 2,4,5,6-tetraaminopyrimidine, p-aminophenol, N,N-bis-(2-hydroxyethyl)-p-phenylenediamine, 2-(2,5-diaminophenyl)-ethanol, 2-(2,5-diaminophenoxy)-ethanol, 1-phenyl-3-carboxyamido-4-amino-5-pyrazolone, 4-amino-3-methylphenol, 2-aminomethyl-4-aminophenol, 2-hydroxy-4,5,6-triaminopyrimidine, 2,4-dihydroxy-5,6-diaminopyrimidine, 2,5,6-triaminohydroxy pyrimidine and 1,3-N,N′-bis-(2′-hydroxyethyl)-N,N′-bis-(4′-aminophenyl)-diamino-2-propanol.

The secondary intermediates are generally m-phenylenediamine derivatives, naphthols, resorcinol and resorcinol derivatives, pyrazolones and m-aminophenols. Particularly suitable secondary intermediates are 1-naphthol, 1,5-, 2,7- and 1,7-dihydroxynaphthalene, 5-amino-2-methylphenol, m-aminophenol, resorcinol, resorcinol monomethyl ether, m-phenylenediamine, 1-phenyl-3-methyl-5-pyrazolone, 2,4-dichloro-3-aminophenol, 1,3-bis-(2,4-diaminophenoxy)-propane, 2-chlororesorcinol, 4-chlororesorcinol, 2-chloro-6-methyl-3-aminophenol, 2-amino-4-hydroxypyridine, 2-methyl resorcinol, 5-methyl resorcinol and 2-methyl-4-chloro-5-aminophenol.

In general, natural color tones cannot be obtained with a primary intermediate alone or with a special secondary intermediate/primary intermediate combination. In practice, therefore, combinations of various primary intermediates and secondary intermediates are used so that there is a constant need for new improved dye components.

Accordingly, the problem addressed by the present invention was to provide new primary intermediates which would satisfy the requirements oxidation dye precursors are expected to meet to a particular degree.

### SUMMARY OF THE INVENTION

It has now been found that certain, hitherto unknown p-aminophenol derivatives satisfy the requirements primary intermediates are expected to meet to a particularly high degree. Thus, brilliant color tones, more especially in the brown and red range, are obtained using these primary intermediates with most of the known secondary intermediates. In addition, the colors obtained are distinguished by extremely high fastness to light and washing.

### DETAILED DESCRIPTION OF THE INVENTION

In a first embodiment, therefore, the present invention relates to p-aminophenol derivatives corresponding to general formula (I):

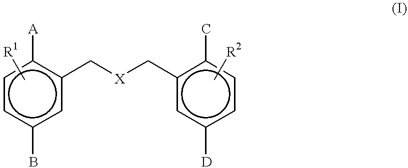

in which

A, B, C and D independently of one another represent an —OH or —NHR group where R is a hydrogen atom or a C₁₋₄ alkyl group, with the proviso that either A or B and C or D is a hydroxy group,

X stands for oxygen, sulfur, a sulfoxy or a sulfoxyl group and

R¹ and R² independently of one another represent hydrogen, fluorine, chlorine, a C₁₋₄ alkyl or hydroxyalkyl group or a C₂₋₄ dihydroxyalkyl group, preferably a C₂ dihydroxyalkyl group.

These compounds can be produced by known organic synthesis methods. Particulars can be found in the following Synthesis Example.

Since all the compounds according to the invention are amino compounds, the known acid addition salts can be prepared from them in the usual way. Accordingly, all statements in this specification and hence the claimed scope of protection relate both to the p-aminophenol derivatives of formula (I) present in free form and to their water-soluble physiologically compatible salts. Examples of such salts are the hydrochlorides, the hydrobromides, the sulfates, the phosphates, the acetates, the propionates, the citrates and the lactates.

Those aminophenol derivatives of formula (I) where X is an oxygen or sulfur atom have proved to be particularly suitable for the purposes of the invention. Oxygen has proved to be a particularly advantageous group X.

Those p-aminophenol derivatives corresponding to formula (I) where the groups A, B, C and D which are not OH groups stand for —NH₂ groups are also particularly preferred for the purposes of the invention.

In addition, hydrogen has proved to be a particularly suitable substituent R¹ and R² in p-aminophenol derivatives corresponding to formula (I).

Finally, those p-aminophenol derivatives corresponding to formula (I) where R¹ and R² are the same, A and C are the same and B and D are the same have also proved to be particularly suitable for the purposes of the invention.

A compound with particularly outstanding suitability for the purposes of the invention is 4-amino-2-(((5-amino-2-hydroxyphenyl)-methoxy)-methyl)-phenol.

In a second embodiment, the present invention relates to the use of the above-mentioned p-aminophenol derivatives as primary intermediates in oxidation hair colorants.

Finally, in a third embodiment, the present invention relates to oxidation colorants for coloring keratin fibers containing secondary intermediates and primary intermediates in a water-containing carrier, characterized in that one of the above-mentioned p-aminophenol derivatives is present as the primary intermediate.

Keratin fibers in the context of the present invention include pelts, wool, feathers and, more particularly, human hair. Although the oxidation colorants according to the invention are particularly suitable for coloring keratin fibers, there is basically nothing to prevent them from being used in other fields, particularly in color photography.

The oxidation colorants according to the invention contain the primary intermediates corresponding to formula (I) and secondary intermediates and, if desired, may contain other primary intermediates and secondary intermediates.

According to the invention, preferred other primary intermediates are p-phenylenediamine, p-toluylenediamine, p-aminophenol, 2-(2,5-diaminophenyl)-ethanol, N,N-bis-(2-hydroxyethyl)-p-phenylenediamine, 1-phenyl-3-carboxyamido-4-amino-5-pyrazolone, 4-amino-3-methylphenol, 4-amino-3-fluorophenol, 2-aminomethyl-4-aminophenol, 2,4,5,6-tetraaminopyrimidine, 2-hydroxy-4,5,6-triaminopyrimidine, 4-hydroxy-2,5,6-triaminopyrimidine, 2,4-dihydroxy-5,6-diaminopyrimidine, 2-dimethylamino-4,5,6-triaminopyrimidine, 2-hydroxyethylaminomethyl-4-aminophenol and 4,4′-diaminodiphenylamine. 2-hydroxymethyl-4-aminophenol, bis-(2-hydroxy-5-aminophenyl)-methane, 1,4-bis-(4-aminophenyl)-diazacycloheptane, 1,3-N,N′-bis-(2′-hydroxyethyl)-N,N′-(4′-aminophenyl)-diamino-1,3-propan-2-ol and 4,5-diaminopyrazole derivatives according to EP 0 740 741 or WO 94108970, such as 4,5-diamino-1-(2′-hydroxyethyl)-pyrazole for example, are also primary intermediates which may advantageously be combined with the primary intermediates according to the invention.

Most particularly preferred other primary intermediates are p-phenylenediamine, 2,4,5,6-tetraminopyrimidine, 4,5-diamino-1-(2′-hydroxyethyl)-pyrazole, N,N-bis-(2-hydroxyethyl)-p-phenylenediamine, bis-(2-hydroxy-5-aminophenyl)-methane, 2-(2,5-diaminophenyl)-ethanol, 3-methyl-4-aminophenol, 4-hydroxy-2,5,6-triaminopyrimidine, 2-aminomethyl-4-aminophenol and p-toluylenediamine.

According to the invention, preferred secondary intermediates are 1-naphthol, pyrogallol, 1,5-, 2,7- and 1,7-dihydroxynaphthalene, o-aminophenol, 5-amino-2-methylphenol, m-aminophenol, resorcinol, resorcinol monomethyl ether, 1-phenyl-3-methyl-5-pyrazolone, 2,4-dichloro-3-aminophenol, 1,3-bis-(2,4-diaminophenoxy)-propane, 4-chlororesorcinol, 2-chloro-6-methyl-3-aminophenol, 2-methyl resorcinol, 5-methyl resorcinol, 2,5-dimethyl resorcinol, 2,6-dihydroxypyridine, 2,6-diaminopyridine, 2-amino-3-hydroxypyridine, 2,6-dihydroxy-3,4-diaminopyridine, 2-methyl-4-chloro-5-aminophenol, 6-methyl-1,2,3,4-tetrahydroquinoxaline, 3-amino-2-methylamino-6-methoxypyridine, 4-amino-2-hydroxytoluene, 2,6-bis-(2-hydroxyethylamino)-toluene, 3.4-methylenediioxyphenol, 3,4-methylenedioxyaniline, 2,4-diaminophenoxyethanol, 2-amino-4-(2-hydroxyethylamino)-anisole and 2,6-dimethyl-3-aminphenol.

Particularly preferred secondary intermediates are 1-naphthol, m-aminophenol, 5-amino-2-methylphenol, 3-methylsulfonylamino-2-methylaniline, 3-amino-2-methylamino-6-methoxypyridine, 2,6-dimethyl-3-aminophenol, 2,4-diaminophenoxy ethanol, 2,7-dihydroxynaphthalene, 6-methyl-1,2,3,4-tetrahydroquinoxaline, 1,7-dihydroxynaphthalene, 2-methyl-4-chloro-5-aminophenol, 3,4-methylenedioxyaniline, 2-methylresorcinol, 4-chlororesorcinol, 3,4-methylenedioxyphenol, 2-amino-3-hydroxypyridine and 2-chloro-6-methyl-3-aminophenol.

These other primary and secondary intermediates are normally used in free form. Where they contain amino groups, however, it may be preferable to use them in salt form, particularly in the form of the hydrochlorides and sulfates.

The hair colorants according to the invention contain both the primary intermediates and the secondary intermediates in a quantity of preferably 0.005 to 20% by weight and more preferably 0.1 to 5% by weight, based on the oxidation colorant as a whole. The primary intermediates and secondary intermediates are generally used in a substantially equimolar ratio to one another. Although it has proved to be of advantage to use the primary and secondary intermediates in an equimolar ratio, there is no disadvantage in using individual oxidation dye precursors in a certain excess so that primary intermediates and secondary intermediates may be present in a molar ratio of 1:0.5 to 1:3 and, more particularly, 1:1 to 1:2.

In one preferred embodiment, the hair colorants according to the invention contain typical substantive dyes in addition to the oxidation dye precursors for further modifying the color tones. Substantive dyes are normally nitrophenylendiamines, nitroaminophenols, azo dyes, anthraquinones or indophenols. Preferred substantive dyes are the compounds known under the International names or trade names of HC Yellow 2, HC Yellow 4, HC Yellow 6, Basic Yellow 57, Disperse Orange 3, HC Red 3, HC Red BN, Basic Red 76, HC Blue 2, Disperse Blue 3, Basic Blue 99, HC Violet 1, Disperse Violet 1, Disperse Violet 4, Disperse Black 9, Basic Brown 16 and Basic Brown 17 and also 4-amino-2-nitrodiphenylamine-2′-carboxylic acid, 6-nitro-1,2,3,4-tetrahydroquinoxaline, hydroxyethyl-2-nitrotoluidine, picramic acid, 2-amino-6-chloro-4-nitrophenol and 4-N-ethyl-1,4-bis-(2′-hydroxyethylamino)-2-nitrobenzene hydrochloride. The colorants according to the invention of this embodiment preferably contain the substantive dyes in a quantity of 0.01 to 20% by weight, based on the colorant as a whole.

The colorants according to the invention may also contain naturally occurring dyes such as, for example, henna red, henna neutral, henna black, camomile blossom, sandalwood, black tea, black alder bark, sage, logwood, madder root, catechu, sedre and alkanet.

Other dye components which may be present in the colorants according to the invention are indoles and indolines and physiologically compatible salts thereof. Preferred examples are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 6-hydroxyindole, 6-aminoindole and 4-aminoindole. Also preferred are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 6-hydroxyindoline, 6-aminoindoline and 4-aminoindoline.

The oxidation dye precursors or the substantive dyes optionally present do not have to be single compounds. On the contrary, other components may be present in small quantities in the hair colorants according to the invention due to the processes used to produce the individual dyes providing these other components do not adversely affect the coloring result or have to be ruled out for other reasons, for example toxicological reasons.

So far as the dyes suitable for use in the hair colorants and tinting formulations according to the invention are concerned, reference is also expressly made to the work by Ch. Zviak, The Science of Hair Care, Chapter 7 (pages 248-250; substantive dyes) and Chapter 8, pages 264-267; oxidation dye precursors), published as Volume 7 of the Series “Dermatology” (Ed.: Ch. Culnan and H. Maibach), Marcel Dekker Inc., New York/Basle, 1986, and to the “Europäische Inventar der Kosmetik-Rohstoffe” published by the Europaische Gemeinschaft and available in disk form from the Bundesverband Deutscher Industrie- und Handelsunternehmen für Arzneimittel, Reformwaren und Körperpflegemittel d.V., Mannheim.

To produce the colorants according to the invention, the oxidation dye precursors are incorporated in a suitable water-containing carrier. For coloring hair, such carriers are, for example, creams, emulsions, gels or even surfactant-containing foaming solutions, for example shampoos, foam aerosols or other formulations suitable for application to the hair.

The colorants according to the invention may also contain any of the known active substances, additives and auxiliaries typical of such formulations. In many cases, the colorants contain at least one surfactant, both anionic and zwitterionic, ampholytic, nonionic and cationic surfactants being suitable in principle. In many cases, however, it has been found to be of advantage to select the surfactants from anionic, zwitterionic or nonionic surfactants.

Suitable anionic surfactants for the hair colorants according to the invention are any anionic surface-active substances suitable for use on the human body. Such substances are characterized by a water-solubilizing anionic group such as, for example, a carboxylate, sulfate, sulfonate or phosphate group and a lipophilic alkyl group containing around 10 to 22 carbon atoms. In addition, glycol or polyglycol ether groups, ester, ether and amide and hydroxyl groups may also be present in the molecule. The following are examples of suitable anionic surfactants —in the form of the sodium, potassium and ammonium salts and the mono-, di- and trialkanol-ammonium salts containing 2 or 3 carbon atoms in the alkanol group:

linear fatty acids containing 10 to 22 carbon atoms (soaps),

ether carboxylic acids corresponding to the formula R—O—(CH₂—CH₂O)ₓ—CH₂—COOH, in which R is a linear alkyl group containing 10 to 22 carbon atoms and x=0 or 1 to 16,

acyl sarcosides containing 10 to 18 carbon atoms in the acyl group,

acyl taurides containing 10 to 18 carbon atoms in the acyl group,

acyl isethionates containing 10 to 18 carbon atoms in the acyl group,

sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups,

linear alkane sulfonates containing 12 to 18 carbon atoms,

linear α-olefin sulfonates containing 12 to 18 carbon atoms,

α-sulfofatty acid methyl esters of fatty acids containing 12 to 18 carbon atoms,

alkyl sulfates and alkyl polyglycol ether sulfates corresponding to the formula R—O(CH₂—CH₂O)ₓ—SO₃H, in which R is a preferably linear alkyl group containing 10 to 18 carbon atoms and x=0 or 1 to 12,

mixtures of surface-active hydroxysulfonates according to DE-A-37 25 030,

sulfated hydroxyalkyl polyethylene and/or hydroxyalkylene propylene glycol ethers according to DE-A-37 23 354,

sulfonates of unsaturated fatty acids containing 12 to 24 carbon atoms and 1 to 6 double bonds according to DE-A-39 26 344,

esters of tartaric acid and citric acid with alcohols in the form of addition products of around 2 to 15 molecules of ethylene oxide and/or propylene oxide with fatty alcohols containing 8 to 22 carbon atoms.

Preferred anionic surfactants are alkyl sulfates, alkyl polyglycol ether sulfates and ether carboxylic acids containing 10 to 18 carbon atoms in the alkyl group and up to 12 glycol ether groups in the molecule and, in particular, salts of saturated and, more particularly, unsaturated C₈₋₂₂ carboxylic acids, such as oleic acid, stearic acid, isostearic acid and palmitic acid.

In the context of the invention, zwitterionic surfactants are surface-active compounds which contain at least one quaternary ammonium group and at least one —COO⁽⁻⁾ or —SO₃⁽⁻⁾ group in the molecule. Particularly suitable zwitterionic surfactants are the so-called betaines, such as N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. A preferred zwitterionic surfactant is the fatty acid amide derivative known by the CTFA name of Cocamidopropyl Betaine.

Ampholytic surfactants are surface-active compounds which, in addition to a C₈₋₁₈ alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids containing around 8 to 18 carbon atoms in the alkyl group. Particularly preferred ampholytic surfactants are N-cocoalkyl aminopropionate, cocoacyl aminoethyl aminopropionate and C₁₂₋₁₈ acyl sarcosine.

Nonionic surfactants contain, for example, a polyol group, a polyalkylene glycol ether group or a combination of polyol and polyglycol ether groups as the hydrophilic group. Examples of such compounds are

products of the addition of 2 to 30 moles of ethylene oxide and/or 0 to 5 moles of propylene oxide onto linear fatty alcohols containing 8 to 22 carbon atoms, onto fatty acids containing 12 to 22 carbon atoms and onto alkylphenols containing 8 to 15 carbon atoms in the alkyl group,

C₁₂₋₂₂ fatty acid monoesters and diesters of products of the addition of 1 to 30 moles of ethylene oxide onto glycerol,

C₈₋₂₂ alkyl mono- and oligoglycosides and ethoxylated analogs thereof,

products of the addition of 5 to 60 moles of ethylene oxide onto castor oil and hydrogenated castor oil,

products of the addition of ethylene oxide onto sorbitan fatty acid esters,

products of the addition of ethylene oxide onto fatty acid alkanolamides.

Examples of cationic surfactants suitable for use in the hair treatment formulations according to the invention are, in particular, quaternary ammonium compounds. Preferred quaternary ammonium compounds are ammonium halides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride. Other cationic surfactants suitable for use in accordance with the invention are the quaternized protein hydrolyzates.

Also suitable for use in accordance with the invention are cationic silicone oils such as, for example, the commercially available products Q2-7224 (manufacturer: Dow Corning; a stabilized trimethyl silyl amodimethicone), Dow Corning 929 Emulsion (containing a hydroxylamino-modified silicone which is also known as Amodimethicone), SM-2059 (manufacturer: General Electric), SLM-55067 (manufacturer: Wacker) and Abil®-Quat 3270 and 3272 (manufacturer: Th. Goldschmidt; diquaternary polydimethyl siloxanes, Quaternium-80).

Alkyl amidoamines, particularly fatty acid amidoamines, such as the stearyl amidopropyl dimethyl amine obtainable as Tego Amid®S 18, are distinguished not only by their favorable conditioning effect, but also and in particular by their ready biodegradability.

Quaternary ester compounds, so-called “esterquats”, such as the methyl hydroxyalkyl dialkoyloxyalkyl ammonium methosulfates marketed under the trade name of Stepantex® and the products marketed under the trade name of Dehyquart®, such as Dehyquart AU-46, are also readily biodegradable.

One example of a quaternary sugar derivative suitable for use as a cationic surfactant is the commercially available product Glucquat®100 (INCI name: Lauryl Methyl Gluceth-10 Hydroxypropyl Dimonium Chloride).

The compounds containing alkyl groups used as surfactants may be single compounds. In general, however, these compounds are produced from native vegetable or animal raw materials so that mixtures with different alkyl chain lengths dependent upon the particular raw material are obtained.

The surfactants representing addition products of ethylene and/or propylene oxide with fatty alcohols or derivatives of these addition products may be both products with a “normal” homolog distribution and products with a narrow homolog distribution. Products with a “normal” homolog distribution are mixtures of homologs which are obtained in the reaction of fatty alcohol and alkylene oxide using alkali metals, alkali metal hydroxides or alkali metal alcoholates as catalysts. By contrast, narrow homolog distributions are obtained when, for example, hydrotalcites, alkaline earth metal salts of ether carboxylic acids, alkaline earth metal oxides, hydroxides or alcoholates are used as catalysts. The use of products with a narrow homolog distribution can be of advantage.

Other active substances, auxiliaries and additives are, for example,

nonionic polymers such as, for example, vinyl pyrrolidone/vinyl acrylate copolymers, polyvinyl pyrrolidone and vinyl pyrrolidone/vinyl acetate copolymers and polysiloxanes,

cationic polymers, such as quaternized cellulose ethers, polysiloxanes containing quaternary groups, dimethyl diallyl ammonium chloride polymers, acrylamide/dimethyl diallyl ammonium chloride copolymers, dimethyl aminoethyl methacrylate/vinyl pyrrolidone copolymers quaternized with diethyl sulfate, vinyl pyrrolidone/imidazolinium methochloride copolymers and quaternized polyvinyl alcohol,

zwitterionic and amphoteric polymers such as, for example, acrylamidopropyl/trimethyl ammonium chloride/acrylate copolymers and octyl acrylamide/methyl methacrylate/tert.butyl aminoethyl methacrylate/2-hydroxypropyl methacrylate copolymers,

anionic polymers such as, for example, polyacrylic acids, crosslinked polyacrylic acids, vinyl acetate/crotonic acid copolymers, vinyl pyrrolidonelvinyl acrylate copolymers, vinyl acetate/butyl maleate/isobornyl acrylate copolymers, methyl vinyl ether/maleic anhydride copolymers and acrylic acid/ethyl acrylate/N-tert.butyl acrylamide terpolymers,

thickeners, such as agar agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives, such as amylose, amylopectin and dextrins, clays such as, for example, bentonite or fully synthetic hydrocolloids such as, for example, polyvinyl alcohol,

structurants, such as glucose and maleic acid,

hair-conditioning compounds, such as phospholipids, for example soya lecithin, egg lecithin and kephalins, and also silicone oils,

protein hydrolyzates, more particularly elastin, collagen, keratin, milk protein, soya protein and wheat protein hydrolyzates, condensation products thereof with fatty acids and quaternized protein hydrolyzates,

perfume oils, dimethyl isosorbide and cyclodextrins,

solubilizers, such as ethanol, isopropanol, ethylene glycol, propylene glycol, glycerol and diethylene glycol,

antidandruff agents, such as piroctone olamine and zinc omadine,

other substances for adjusting the pH value,

active substances, such as panthenol, pantothenic acid, allantoin, pyrrolidone carboxylic acids and salts thereof, plant extracts and vitamins,

cholesterol,

UV filters,

consistency factors, such as sugar esters, polyol esters or polyol alkyl ethers,

fats and waxes, such as spermaceti, beeswax, montan wax, paraffins, fatty alcohols and fatty acid esters,

fatty acid alkanolamides,

complexing agents, such as EDTA, NTA and phosphonic acids,

swelling and penetration agents, such as glycerol, propylene glycol monoethyl ether, carbonates, hydrogen carbonates, guanidines, ureas and primary, secondary and tertiary phosphates,

opacifiers, such as latex,

pearlizers, such as ethylene glycol mono- and distearate,

propellents, such as propane/butane mixtures, N₂O, dimethyl ether, CO₂ and air and

antioxidants.

To produce the colorants according to the invention, the constituents of the water-containing carrier are used in the usual quantities for this purpose. For example, emulsifiers are used in concentrations of 0.5 to 30% by weight while thickeners are used in concentrations of 0.1 to 25% by weight, based on the colorant as a whole.

In principle, the color can be oxidatively developed with atmospheric oxygen. However, a chemical oxidizing agent is preferably used, particularly when human hair is to be not only colored, but also lightened. Particularly suitable oxidizing agents are persulfates, chlorites and, in particular, hydrogen peroxide or addition products thereof with urea, melamine or sodium borate. Oxidation may also be carried out with enzymes. In this case, the enzymes may be used to transfer atmospheric oxygen to the primary intermediate or to enhance the effect of an oxidizing agent present in small quantities. One example of an enzymatic process is the procedure whereby the effect of small quantities (for example 1% and less, based on the formulation as a whole) of hydrogen peroxide is enhanced by peroxidases.

The preparation of the oxidizing agent is preferably mixed with the preparation of the oxidation dye precursors immediately before coloring of the hair. The ready-to-use hair coloring preparation formed should preferably have a pH value in the range from 6 to 10. In a particularly preferred embodiment, the hair colorant is used in a mildly alkaline medium. The application temperatures may be in the range from 15 to 40° C. After a contact time of about 5 to 30 minutes, the hair colorant is removed from the hair to be colored by rinsing. There is no need for the hair to be washed with a shampoo where a carrier of high surfactant content, for example a coloring shampoo, has been used.

The following Examples are intended to illustrate the invention.

### Examples

1. Synthesis Example

Synthesis of 4-amino-2-(((5-amino-2-hydroxyphenyl)-methoxy)-methyl)-phenol

Diazonium solution (solution A):

10.35 g (0.115 mole) of sodium nitrite in 125 ml of water were added at a temperature of +5° C. to a solution consisting of 26 g (0.15 mole) of sulfanilic acid and 75 ml (0.15 mole) of 2N sodium hydroxide. 131 ml (0.38 mole) of 10% hydrochloric acid were added dropwise while cooling (+5° C.) over a period of 30 minutes.

Solution A was added dropwise to an ice-cooled solution consisting of 17.25 g (0.075 mole) of bis-(2-hydroxybenzyl)-ether in 120 ml (0.3 mole) of 10% sodium hydroxide. After the addition, the whole was stirred for 1.5 hours at 20° C.

69 g (0.4 mole) of sodium dithionite were then added over a period of 2 minutes. After 20 minutes at 75° C., the product was filtered off under suction and washed with water. The product was obtained in the form of colorless crystals with a melting point of 175° C. (decomp.).

2. Coloring

A cream base with the following composition was first prepared [all quantities are in g, unless otherwise indicated]:

| | |
|---|---|
| tallow fatty alcohol | 17.0 |
| Lorol ®techn.¹ | 4.0 |
| Texapon ®N 28² | 40.0 |
| Dehyton ®K³ | 25.0 |
| Eumulgin ®B 2⁴ | 1.5 |
| distilled water | 12.5 |

¹C₁₂₋₁₈ fatty alcohol (HENKEL)
²Sodium lauryl ether sulfate (ca. 28% active substance; INCI name: Sodium Laureth Sulfate) (HENKEL)
³Fatty acid amide derivative with a betaine structure corresponding to the following formula: R—CONH(CH₂)₃N⁺(CH₃)₂CH₂COO⁻(ca. 30% active substance; INCI name Cocoamidopropyl Betaine) (HENKEL)
⁴Cetyl stearyl alcohol containing ca. 20 moles EO (INCI name: Ceteareth-20) (HENKEL)

¹ C₁₂₋₁₈ fatty alcohol (HENKEL)

² Sodium lauryl ether sulfate (ca. 28% active substance; INCI name: Sodium Laureth Sulfate) (HENKEL)

³ Fatty acid amide derivative with a betaine structure corresponding to the following formula: R—CONH(CH₂)₃N⁺(CH₃)₂CH₂COO⁻ (ca. 30% active substance; INCI name Cocoamidopropyl Betaine) (HENKEL)

⁴ Cetyl stearyl alcohol containing ca. 20 moles EO (INCI name: Ceteareth-20) (HENKEL)

The following hair coloring cream emulsion was then prepared on the basis of this cream:

| | |
|---|---|
| cream base | 50.0 |
| primary intermediate | 7.5 mmoles* |
| secondary intermediate | 7.5 mmoles* |
| Na₂SO₃ (inhibitor) | 1.0 |
| (NH₄)₂SO₄ | 1.0 |
| conc. ammonia solution to pH | 10 |
| water to | 100 |

*unless otherwise indicated

The ingredients were mixed in the order listed. Substantive dyes were optionally added with corresponding reductions in the water content. After addition of the oxidation dye precursors and the inhibitor, the emulsion was first adjusted to pH 10 with concentrated ammonia solution and was then made up with water to 100 g.

The color was oxidatively developed with 3% hydrogen peroxide solution as the oxidizing solution. To this end, 50 g of hydrogen peroxide solution (3%) were added to and mixed with 100 g of the emulsion.

The coloring cream was applied to approximately 5 cm long tresses of standardized, 90% grey but not specially pretreated human hair and left thereon for 30 minutes at 32° C. On completion of the coloring process, the hair was rinsed, washed with a normal shampoo and dried. The following primary and secondary intermediates and substantive dyes were used for coloring:

Primary intermediates

4-amino-2-(((5-amino-2-hydroxyphenyl)-methoxy)-methyl)-phenol (E1)

2,4,5,6-tetraaminopyrimidine (E2)

4,5-diamino-1-(2-hydroxyethyl)-pyrazole (E3)

p-phenylenediamine

N,N-bis-(2-hydroxyethyl)-p-phenylenediamine (E5)

4-hydroxy-2,5,6-triaminopyrimidine (E6)

p-toluylenediamine

2-(2,5-diaminophenyl)-ethanol (E8)

2-aminomethyl-4-aminophenol (E9)

4-amino-3-methylphenol

bis-(2-hydroxy-5-aminophenyl)-methane (E11)

Secondary intermediates

1-naphthol (K1)

2-methyl-5-aminophenol (K2)

2-chloro-6-methyl-3-aminophenol (K3)

2-amino-3-hydroxypyridine (K4)

2,6-dimethyl-3-aminophenol (K5)

m-aminophenol (K6)

2,7-dihydroxynaphthalene (K7)

4-chlororesorcinol (K8)

2-methyl-4-chloro-5-aminophenol (K9)

6-methyl-1,2,3,4-tetrahydroquinoxaline (K10)

2-methyl resorcinol (K11)

3-amino-2-methylamino-6-methoxypyridine (K12)

2,4-diaminophenoxyethanol (K13)

3-methylsulfonylamino-2-methyl aniline (K14)

1,7-dihydroxynaphthalene (K15)

3,4-methylenedioxyphenol (K16)

3,4-methylenedioxyaniline (K17)

m-phenylenediamine (K18)

Substantive dyes

6-nitro-1,2,3,4-tetrahydroquinoxaline (D1)

2-nitro-4-aminodiphenylamine-2′-carboxylic acid (D2)

2-amino-6-chloro-4-nitrophenol (D3)

2-nitro-1-amino-4-bis-(2-hydroxyethyl)-aminobenzene (D4)

2-nitro-1-(2-hydroxyethyl)-amino-4-methylbenzene (D5)

2-(2′-hydroxyethylamino)-4,6-dinitrobenzene (D6)

1-(2′-hydroxyethyl)-amino-2-nitro-4-bis-(2-hydroxyethyl)-aminobenzene (D7)

1-(2-hydroxyethyl)-amino-2-nitro-4-aminobenzene (D8)

4-(2-nitrophenyl)-aminophenol (D9)

The following colors were obtained:

| Primary | Secondary | | |
|---|---|---|---|
| intermediate | intermediate | Substantive dye | Hair color |
| E1 | K1 | — | Grey-red |
| E1 | K2 | — | Grey-orange |
| E1 | K3 | — | Brown-red |
| E1 | K4 | — | Light orange |
| E1 | K5 | — | Grey-red |
| E1 | K6 | — | Topaz yellow |
| E1 + E3^{a} | K1 + K3^{a} | — | Old pink |
| E1 + E2^{a} | K2 + K7^{a} | — | Flat violet |
| E1 + E6^{a} | K8 + K9^{a} | — | Grey-red |
| E1 + E7^{a} | K10 + K11^{a} | — | Champagne |
| E1 + E4^{a} | K12 | — | Black |
| E1 + E5^{a} | K5 + K13^{a} | — | Black-blue |
| E1 + E8^{a} | K12 + K14^{a} | — | Blue-grey |
| E1 + E9^{a} | K15 + K16^{a} | 0.1% by weight D1 | Brown-orange |
| E1 + E10^{a} | K17 | 0.3% by weight D2 | Flat red |
| E1 + E11^{a} | K18 | 0.1% by weight D3 | Hair brown |
| E1 | K3 | 0.1% by weight D4 + 0.1% | Copper |
| | | by weight D5 | |
| E1 | K3 | 0.1% by weight D6 + 0.1% | Tomato red |
| | | by weight D7 | |
| E1 | K9 | 0.1% by weight D8 + 0.1% | Lacquer red |
| | | by weight D9 | |

^{a}0.375 mmole

What is claimed is:

1. p-Aminophenol derivatives corresponding to general formula (I): 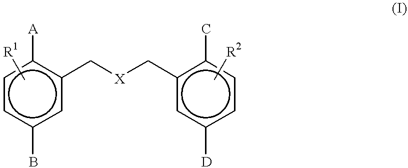
in which
A, B, C and D independently of one another represent an —OH or —NHR group where R is a hydrogen atom or a C₁₋₄ alkyl group, with the proviso that either A or B and C or D is a hydroxy group,
X stands for oxygen, sulfur, a sulfoxy or a sulfoxyl group, and
R¹ and R² independently of one another represent hydrogen, fluorine, chlorine, a C₁₋₄ alkyl or hydroxyalkyl group or a C₂₋₄ dihydroxyalkyl group;
or physiologically compatible salts of these compounds.

2. The p-aminophenol derivatives of claim 1, wherein X stands for an oxygen or sulfur atom.

3. The p-aminophenol derivatives of claim 2, wherein X stands for an oxygen atom.

4. The p-aminophenol derivatives of claim 1, wherein the groups A, B, C, and D that are not —OH groups are —NH₂ groups.

5. The p-aminophenol derivatives of claim 1, wherein R¹ and R² represent hydrogen.

6. The p-aminophenol derivatives of claim 1, wherein the groups R¹ and R² are the same, A and C are the same, and B and D are the same.

7. The p-aminophenol derivatives of claim 1 wherein the p-aminophenol derivatives comprise 4-amino-2-(((5-amino-2-hydroxyphenyl)-methoxy)-methyl)-phenol.

8. A method for coloring keratin fibers comprising contacting said fibers with a coloring composition comprising a coloring-effective amount of one or more of the p-aminophenol derivatives as claimed in claim 1 or a physiologically compatible salt thereof, and oxidatively developing the coloring composition on said fibers.

9. An oxidation colorant for coloring keratin fibers comprising, in a water-containing carrier, one or more secondary intermediates and one or more primary intermediates, wherein at least one of the primary intermediates is one or more of the p-aminophenol derivatives according to claim 1.

10. The oxidation colorant of claim 9, wherein at least one of the secondary intermediates is selected from the group consisting of 1-naphthol, m-aminophenol, 5-amino-2-methylphenol, 3-methylsulfonylamino-2-methylaniline, 3-amino-2-methylamino-6-methoxypyridine, 2,6-dimethyl-3-aminophenol, 2,4-diaminophenoxy ethanol, 2,7-dihydroxynaphthalene, 6-methyl-1,2,3,4-tetrahydroquinoxaline, 1,7-dihydroxynaphthalene, 2-methyl-4-chloro-5-aminophenol, 3,4-methylenedioxyaniline, 2-methylresorcinol, 4-chlororesorcinol, 3,4-methylenedioxyphenol, 2-amino-3-hydroxypyridine, and 2-chloro-6-methyl-3-aminophenol.

11. The oxidation colorant of claim 9, comprising a second primary intermediate in addition to the one or more p-aminophenol derivatives.

12. The oxidation colorant of claim 11, wherein the second primary intermediate is selected from the group consisting of 2,4,5,6-tetraaminopyrimidine, 4-hydroxy-2,5,6-triaminopyrimidine, 4,5-diamino-1-(2-hydroxyethyl)-pyrazole, p-toluylenediamine, 2-aminomethyl-4-aminophenol, N,N-bis-(2-hydroxyethyl)-p-phenylenediamine, p-phenylenediamine, 2-(2,5-diaminophenyl)-ethanol, 3-methyl-p-aminophenol, bis-(2-hydroxy-5-aminophenyl)-methane and combinations thereof.

13. The oxidation colorant of claim 9, comprising the primary intermediates in a quantity of 0.005% to 20% by weight and the secondary intermediates in a quantity of 0.005% to 20% by weight, based on the total weight of the oxidation colorant.

14. The oxidation colorant of claim 13, comprising the primary intermediates in a quantity of 0.1% to 5% by weight and the secondary intermediates in a quantity of 0.1% to 5% by weight, based on the total weight of the oxidation colorant.

15. The oxidation colorant of claim 9, further comprising at least one substantive dye.

16. A method for coloring keratin fibers comprising:
(a) forming an oxidation dye precursor composition comprising a coloring-effective amount of one or more of the p-aminophenol derivatives as claimed in claim 1, or a physiologically compatible salt thereof;
(b) mixing atmospheric oxygen or an oxidizing agent with the oxidation dye precursor composition to form a colorant; and
(c) contacting the fibers with the colorant.
